(19) United States
(12) Patent Application Publication
(10) Pub. No.: US 2004/0019296 A1
(43) Pub. Date: Jan. 29, 2004

(54) Nestable needle holder and package

(76) Inventor: Volker Niermann, Boundbrook, NJ (US)

Correspondence Address: BECTON, DICKINSON AND COMPANY, 1 BECTON DRIVE, FRANKLIN LAKES, NJ, 07417-1880, US

(21) Appl. No.: 10/207,533

(22) Filed: Jul. 29, 2002

(51) Int. Cl. A61B 10/00

(52) U.S. Cl. 600/562

(57) ABSTRACT

A needle holder including a body having a closed end, an opposed open end, and a tubular wall therebetween. The needle holder is designed to insertably accommodate a specimen collection tube therein. The tubular wall of the needle holder body has an outer diameter adjacent the closed end which is designed to be nestably accommodated within an inner diameter of the open end of an adjacent needle holder body. Plural nestable needle holder bodies may be arranged in an array. Each array may be enclosed in a polybag. Plural such arrays may be packaged in a carton for shipping and storage.

### BACKGROUND OF THE INVENTION

1. Field of Invention

The present invention relates to a nestable needle holder for supporting a specimen collection tube and, more particularly, the present invention relates to a stackable array of needle holders which provide for convenient packaging.

2. Description of Related Art

Blood samples and other medical specimens are routinely taken and collected in a specimen collection container. In blood collection applications, the collection container is typically a hollow blood collection tube with one end closed by a semi-spherical portion and the other end open. The open end is sealable by an impervious elastomeric cover. The tube thus defines an interior chamber for collecting and holding the blood sample.

To collect a blood sample, the tube is used in concert with a tube holding device. This device typically comprises a hollow needle extending through the closed end of a tubular housing. The tubular housing has an opposed open end for accepting the blood collection tube. The hollow needle is inserted through the dermal layer into the lumen of the blood vessel and taps into the circulation system of a subject to direct blood therethrough towards the interior compartment of the holding device. The collection container is inserted through the open end of the holder so that the hollow needle punctures through the cover of the collection container. The interior of the collection container is now in direct communication with the circulation system and, having typically been formed in a vacuum, draws blood through the hollow needle and into the collection container. Once the phlebotomist has drawn enough blood into the collection container, the container may be removed from the holder. The container cover, being of an elastomeric material, reseals the hole made by the needle. The phlebotomist may then, if desired, insert a new collection container into the holder to draw more blood.

As may be appreciated, the use of such needle holders is widespread. Many if not all of the current needle holders are designed for “single use only”, thus increasing the number of needle holders used.

Conventionally, these needle holders are shipped in bulk packages where plural needle holders are loosely contained in a polybag. One or more polybags are enclosed in a carton for shipping and storage. A typical polybag may include 250 needle holders and a typical carton would include four polybags. It may be appreciated that 1,000 loosely packed needle holders occupy a large volume of shipping and storage space.

Furthermore, such loosely packaged needle holders are difficult to use and dispense from the carton and polybag.

It is therefore desirable to provide an improved needle holder which can be packaged and shipped more easily and conveniently, and which requires less space for shipping and storage.

### SUMMARY OF THE INVENTION

The present invention provides an improved needle holder which is stackable to facilitate convenient packaging and shipping. The present invention also provides an array of stackable needle holders and a package for containing such stacked arrays.

In one embodiment, the needle holder includes an elongate body having a generally closed end for supporting a needle, an opposed open end for insertably accommodating a specimen collection tube therein, and a generally tubular wall extending therebetween. The tubular wall has an inner diameter adjacent the open end, and an outer diameter adjacent the closed end. The inner diameter of the open end is greater than the outer diameter of the closed end. Plural holders may be nestably stacked with the closed end of one of the holders being insertable into the open end of another holder.

In another embodiment, the present invention provides a needle holder assembly. The assembly includes a plurality of needle holders, each holder having a generally closed end for supporting a needle, an opposed open end for insertably accommodating a specific collective tube, and a tubular wall therebetween. The tubular wall has an inner diameter adjacent the open end which is greater than an outer diameter of the tubular wall adjacent the closed end. The plurality of needle holders are arranged in a stacked array with the closed end of one needle holder being insertably supported within the open end of an adjacently stacked needle holder.

In a further embodiment, the present invention provides a combination of an array of needle holders and a package which accommodates the array. A plurality of needle holders have a generally closed end for supporting a needle, an opposed open end for insertably accommodating a specimen collective tube, and a tubular wall therebetween. The tubular wall has an inner diameter adjacent the open end which is greater than an outer diameter of the tubular wall adjacent the closed end. The plurality of needle holders are arranged in a stacked array with the closed end of one needle holder by insertably supported within the open end of an adjacently stacked needle holder. A carton is provided for supporting and enclosing the stacked array of needle holder bodies.

### DESCRIPTION OF THE DRAWINGS

FIG. 1 is a front plan view of the nestable needle holder of the present invention.

FIG. 2 is a bottom plan view of the nestable needle holder of FIG. 1.

FIG. 3 is a bottom perspective showing of the nestable needle holder of FIG. 1.

FIG. 4 is a vertical sectional showing of plurality of nestable needle holders of FIG. 1 arranged in stacked fashion.

FIG. 5 is a perspective showing of a stacked array of nestable needle holders.

FIG. 6 shows the stacked array of FIG. 5, enclosed in a polybag.

FIG. 7 is an exploded perspective view of a plurality of stacked arrays contained within a shipping carton.

### DETAILED DESCRIPTION

The present invention provides a needle holder for a medical specimen collection tube. More preferably, the needle holder is used to support a blood collection tube for drawing blood from a patient.

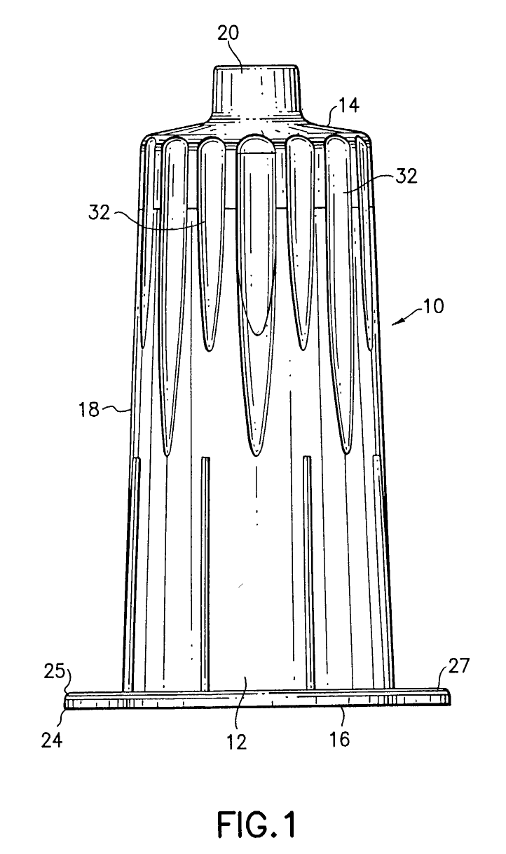

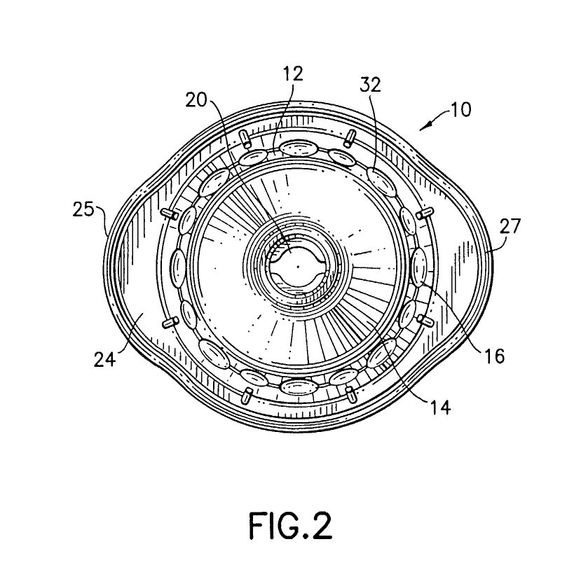

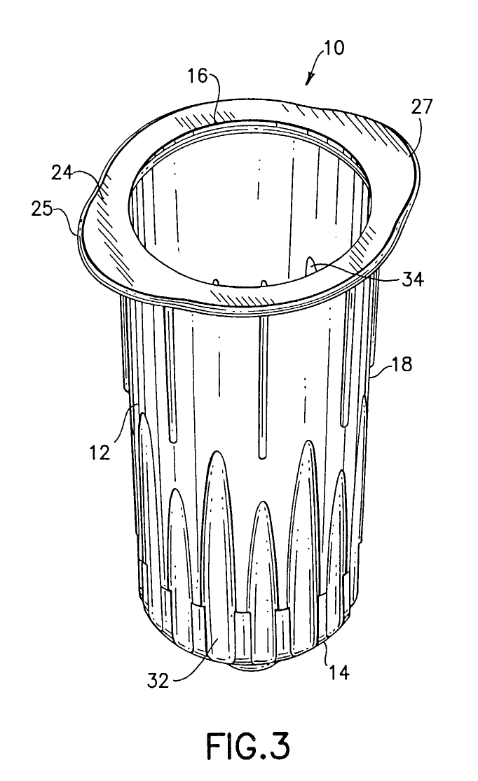

Referring to FIGS. 1-3, needle holder 10 of the present invention is shown. Needle holder 10 includes an elongate body 12 having a closed end 14, an opposed open end 16, and a generally cylindrical wall 18 having an inside surface 19 and an outside surface 21, extending therebetween. Closed end 14 of body 12 includes an extending annular neck 20. Neck 20 includes internal threads 22 which accommodate matching threads on the base of a needle assembly which is attached thereto. Neck 20 may alternatively include other means to attach a needle assembly. Such means include a barbed fitting, full thread or a segmented thread. Open end 16 of body 12 includes a radially extending annular flange 24 extending thereabout. Flange 24 has diametrically opposed flange extension 29 which facilitates manual manipulation of the needle holder.

A needle holder of the type shown in the present invention may be used to accommodate a specimen collection container such as an evacuated blood collection tube. The blood collection tube is inserted through open end 16 of body 12 so that it comes in fluid communication with the needle supported by the neck so as to draw blood from a patient. The filled blood collection tube may be removed and subsequent blood collection tubes may be inserted into the holder to draw additional blood from the patient.

The needle holder of the present invention is contemplated to be a single use device. That is, once the needle holder is used to draw blood from a patient it is not reused. This necessitates that numerous needle holders be on hand for blood collection purposes. The present invention provides a specifically configured needle holder which allows for the stacking of plural needle holders to facilitate space saving in shipping, storage and use.

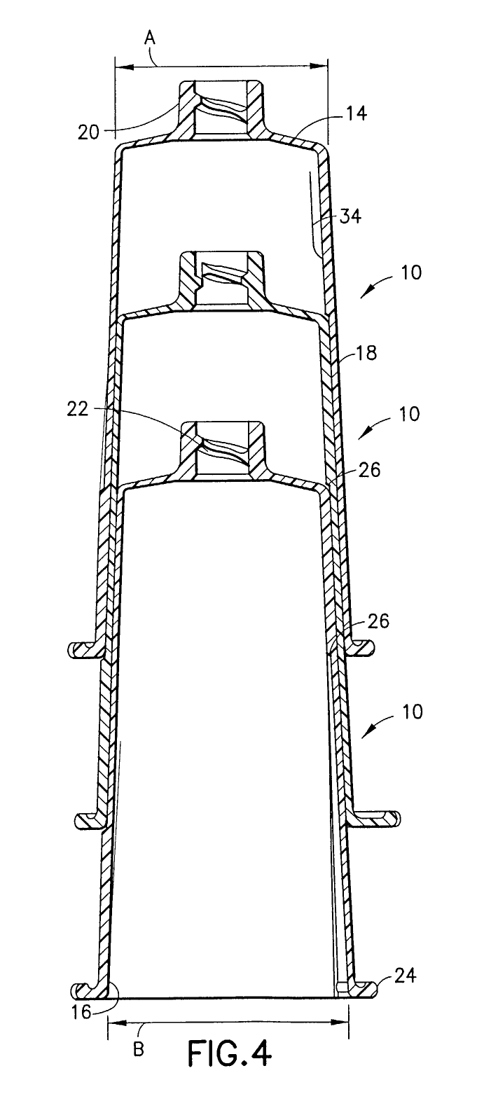

Referring to FIG. 4, needle holder 10 of the present invention is specifically constructed so that cylindrical wall 18 has an outer diameter adjacent to closed end 14 which is less than its inner diameter adjacent open end 16. As shown in FIG. 4, the outer diameter A of the cylindrical wall at the closed end, is less than the inner diameter B of the cylindrical wall adjacent open end 16. This relationship ensures that closed end 14 of body 12 may be insertably accommodated within open end 16 of an adjacent similarly constructed needle holder.

Cylindrical wall 18 may be formed to have a generally slight conical taper, tapering from a wider expanse adjacent open end 16 to a narrower expanse adjacent closed end 14. In addition, it is contemplated that the dimensional relationship between the outer diameter of cylindrical wall 18 adjacent closed end 14, and the inner diameter of cylindrical wall 18 at open end 14 may be achieved by providing a reduction in wall thickness at said open end.

As shown in FIG. 4, the construction of needle holder 10 allows for a plurality of identically formed needle holders to be nested, one inside the other, to define an array 30 of stacked needle holders.

In order to provide structural strength to the needle holder body, the present invention includes a pattern of strengthening ridges 32 adjacent closed end 14. Strengthening ridges 32 provide for increased strength of the needle holder without compromising the wall thickness of cylindrical wall 18. Ridges 32 may also be used to orient the nesting of the needle holders.

In addition, it is possible to include at least one alignment strut or land 34 located about the inside surface of cylindrical wall 18 adjacent open end 16. Alignment strut 34 is an elongated member extending for example from open end 12 centrally towards closed end 14. Alignment strut 34 is capable of orienting the nested needle holders to facilitate stacking. Strut 34 or similar elements may also be employed to align flange extensions 25 and 27 in stacked array 30. Alignment strut 34 may also support and facilitate the stacking and disassembly of the needle holders by providing a clearance between the inner diameter of a front needle holder and the outer diameter of a second needle holder. In addition, alignment strut or land 34 may be formed so that when a blood collection tube is inserted into the needle holder body, the alignment strut is engaged and elastically deformed by the blood collection tube for an increased area of contact with the blood collection tube for holding the tube in the needle holder body. Generally, the alignment strut or land is elastic and flexible.

The shape and construction of cylindrical wall 18 including the provision of ridges 32 may be provided so as to prevent rotation of one needle holder with respect to an adjacently stacked needle holder. This feature also facilitates stacking and dispensing of the needle holder.

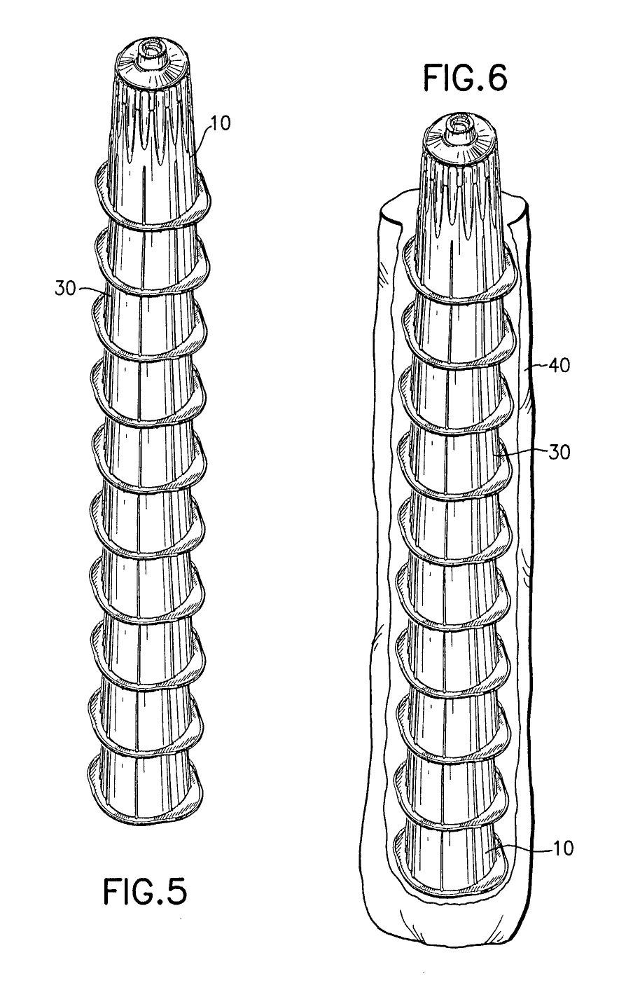

In FIGS. 5, 6 and 7, the arrangement of a plurality of stacked needle holders is arranged. FIG. 5 shows an array 30 of needle holders 10 of the present invention arranged in nested or stacked fashion. Needle holders 10 may be stacked ten in number, however, arrays of more or less needle holders may be stacked. A single array 30 of stacked needle holders may be enclosed in a polybag or sleeve 40. Sleeve 40 helps isolate the stacked needle holder array 30 from debris, and also provides a convenient method for dispensing the individual needle holders. The polybag or sleeve 40 may be formed from a one mil thick ply of polyethylene or polypropylene.

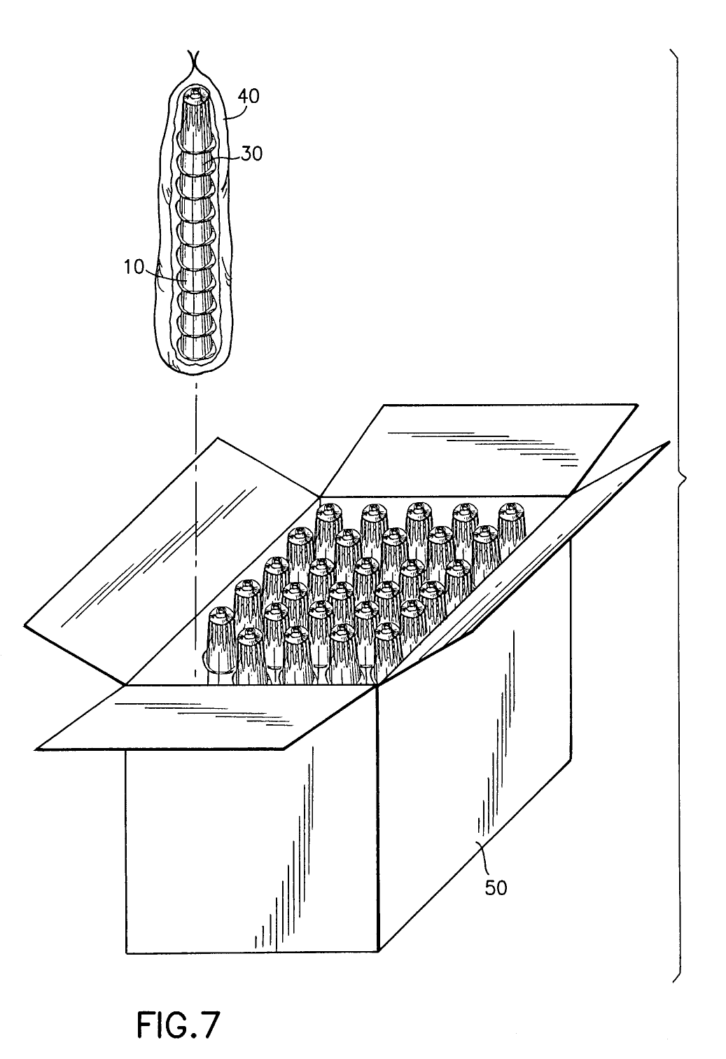

As shown in FIG. 7, plural arrays 30 of needle holders 10 arranged in polybags 40 may be packaged in a shipping carton 50. The shipping carton may be of size and shape to accommodate any number of stacked arrays of needle holders, for example a carton 50 which will accommodate ten stacked arrays of needle holders in a ten-by-ten pattern. Thus, each carton 50 would conveniently and efficiently accommodate 1,000 needle holders in 100 stacked arrays.

While the particular embodiment of the present invention has been shown and described, it will be apparent to those skilled in the art that changes and modifications may be made without departing from the fundamental teachings of the invention. The matter set forth in the forgoing description and accompanying drawings is offered by way of illustration only and not as a limitation. The actual scope of the invention is intended to be defined in the following claims when viewed in their proper perspective based on the prior art.

What is claimed:

1. A needle holder comprising:
an elongate body having a generally closed end for supporting a needle, an opposed open end for insertably accommodating a specimen collection tube therein, and a generally tubular wall having an inner surface and an outer surface and extending therebetween;
said tubular wall having an inner diameter adjacent said open end, and an outer diameter adjacent said closed end, and wherein said inner diameter of said open end is greater than said outer diameter of said closed end;
whereby a first said holder is capable of being nestably stacked with a second said holder by placing said closed end of the first holder into said open end of the second holder.

2. The holder of claim 1, wherein said tubular wall includes a conically inward taper, tapering from said open end to said closed end.

3. The holder of claim 1, wherein said tubular wall has a first wall thickness adjacent said closed end and a second wall thickness adjacent said open end, and wherein said first wall thickness is greater than said second wall thickness.

4. The holder of claim 3, wherein the wall thickness gradually changes between said open end toward said closed end.

5. The holder of claim 1, wherein said closed end of said body includes an extending annular neck for supporting said needle.

6. The holder of claim 1, wherein said tubular wall includes one or more strengthening ridges.

7. The holder of claim 1, wherein said tubular wall includes one or more alignment elements.

8. The holder of claim 1, wherein said inner surface of said tubular wall includes at least one flexible land.

9. An assembly of needle holders comprising:
a plurality of needle holder bodies, each body having a generally closed end for supporting a needle, an opposed open end for insertably accommodating a specimen collection tube, and a tubular wall therebetween, said tubular wall having an inner diameter adjacent said open end which is greater than an outer diameter of said tubular wall adjacent said closed end;
said plurality of needle holder bodies arranged in a stacked array with said closed end of one said needle holders being insertably supported within the open end of an adjacently stacked needle holder.

10. An assembly of claim 9, wherein said tubular wall of each said needle holder body is conically inwardly tapered towards said closed end.

11. An assembly of claim 9, wherein said tubular wall of each said needle holder body has a first wall thickness adjacent said closed end and a reduced wall thickness adjacent said open end.

12. An assembly of claim 11, wherein said stacked array is disposed in a storage article.

13. An assembly of claim 11 wherein said tubular wall includes one or more alignment elements for preventing rotation of one of said needle holders of said stacked array with respect to another.

14. A package comprising:
a plurality of needle holder bodies, each body having a generally closed end for supporting a needle, an opposed open end for insertably accommodating a specimen collection tube, and a tubular wall therebetween, said tubular wall having an inner diameter adjacent said open end which is greater than an outer diameter of said tubular wall adjacent said closed end;
said plurality of needle holder bodies arranged in a stacked array with said closed end of one of said needle holder bodies being insertably supported within the open end of an adjacently stacked needle holder body; and
a carton for supporting and enclosing said stacked array of needle holder bodies.

15. The combination of claim 13, wherein said needle holder bodies are arranged in plural stacked arrays and wherein said carton supports and encloses said plural stacked arrays.

16. The combination of claim 13, wherein each stacked array is encased in a flexible sleeve.

17. The combination of claim 15, wherein said flexible sleeve is a polybag.
